(19) United States
(12) Patent Application Publication
(10) Pub. No.: US 2007/0276283 A1
(43) Pub. Date: Nov. 29, 2007

(54) Clinical thermometer capable of recording and analyzing the day of getting pregnant

(76) Inventor: Chin-Yeh Hung, Chang Pin Town (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/433,440

(22) Filed: May 15, 2006

(51) Int. Cl. A61B 10/00

(52) U.S. Cl. 600/551

(57) ABSTRACT

A clinical thermometer capable of recording and analyzing the day of getting pregnant includes a casing, an LCD device, a press key unit, a body temperature detecting element for detecting a body temperature, a microprocessor for receiving a signal from the body temperature detecting element, computing the signal and displaying the body temperature by the LCD device, a flash memory connected to the microprocessor for storing data and having a partitioned memory block for storing an Auto Run program, and a power supply unit electrically connected to the microprocessor for supplying power to components, and a USB interface installed at the casing and electrically connected to the microprocessor, such that when the USB interface is connected to a personal computer, the Auto Run program automatically sends the body temperature data to the personal computer to facilitate recording and analyzing a better day of getting pregnant.

### BACKGROUND OF THE INVENTION

1. Field of the Invention

The invention relates to a clinical thermometer, and more particularly to a clinical thermometer capable of automatically conveying body temperature data as well as recording and analyzing a better day for getting pregnant.

2. Description of the Related Art

As our life style has changed, it is very common for people to get married late, and many women cannot get pregnant even when they are ready to have babies due to many different factors such as the pressure from our work, and thus such problem disturbs many couples and families.

As to the present medical technology, many contraceptive methods are available and they are simple, easy and effective. However, the way of getting pregnant becomes increasingly difficult, and more and more people rely on artificial pregnancy or have test-tube babies. As we all know that such long and difficult process may not be successful all the time. Regardless of a natural pregnancy or an artificial pregnancy, it is very important to accurately calculate a better day of getting pregnant. At present, there are two main methods for calculating a better day for a woman to get pregnant, and these methods are the menstrual cycle method and the basic body temperature method. For those who use the menstrual cycle method to calculate the best day for getting pregnant, their menstrual cycle must be regular, or else the accuracy of the calculation will be affected.

As to the basic body temperature method, a woman's body temperature will drop slightly at an ovulation day. The body temperature will rise about 0.5° C. after the ovulation due to the influence of progesterone, and such temperature will remain for approximately 14 days until the next period. Therefore, the aforementioned rise of body temperature indicates the ovulation day. However, the basic body temperature method requires users to start measuring their body temperature by a clinical thermometer from the first day of a menstrual cycle and record the data in detail by pen and paper. Such application causes great inconvenience. Furthermore, all data cannot be compiled easily for a graphical analysis or a prediction for the best day of getting pregnant. Thus, the aforementioned methods still require further improvements. SUMMARY OF THE INVENTION

Therefore, it is a primary object of the present invention to provide a clinical thermometer capable of recording and analyzing a better day of getting pregnant that records the body temperature of a user day by day, starting from the first day of a menstrual period of the user, and adopts a USB interface and an Auto Run program to convey the body temperature data to a personal computer for further recording and analysis, so as to predict the best day of getting pregnant.

The invention also provides another advantage that it can analyze a safe period for the contraception purpose.

In order to achieve the above-mentioned object and advantage, a clinical thermometer capable of recording and analyzing the day of getting pregnant, includes:
a) a casing being substantially in a rectangular shape and having a containing space for installing an LCD device and a press key unit at the front side of the casing;
b) a body temperature detecting element installed at a small end of the casing for detecting a body temperature;
c) a microprocessor for receiving a signal transmitted from the body temperature detecting element and computing the body temperature and displaying a numeric data of the body temperature by the LCD device;
d) a flash memory coupled to the microprocessor for storing the data and having a partitioned memory block with at least one area for storing an Auto Run program;
e) a power supply unit electrically coupled to the microprocessor for supplying electric power required by each of the foregoing elements; and
f) a USB interface installed at a side of the casing and electrically coupled to the microprocessor,
   whereby, when the USB interface is coupled to a personal computer, the Auto Run program pre-stored in the flash memory can automatically send the body temperature data accumulated by a user for a predetermined time to an operating system of the personal computer, so as to facilitate recording and analyzing a better day of getting pregnant.

### BRIEF DESCRIPTION OF THE FIGURES

FIG. 1 is a schematic view of a first preferred embodiment of the invention;

FIG. 2 is a schematic view of a second preferred embodiment of the invention;

FIG. 3 is a circuit block diagram of the invention;

FIG. 4 is a schematic view of connecting a clinical thermometer to a personal computer according to the invention;

FIG. 5 is a schematic view of an application of the invention;

FIG. 6 is a schematic view of another application of the invention; and

FIG. 7 is a schematic view of a clinical thermometer with a protective cover according to the invention.

### DETAILED DESCRIPTION OF THE PREFERRED EMBODIMENT

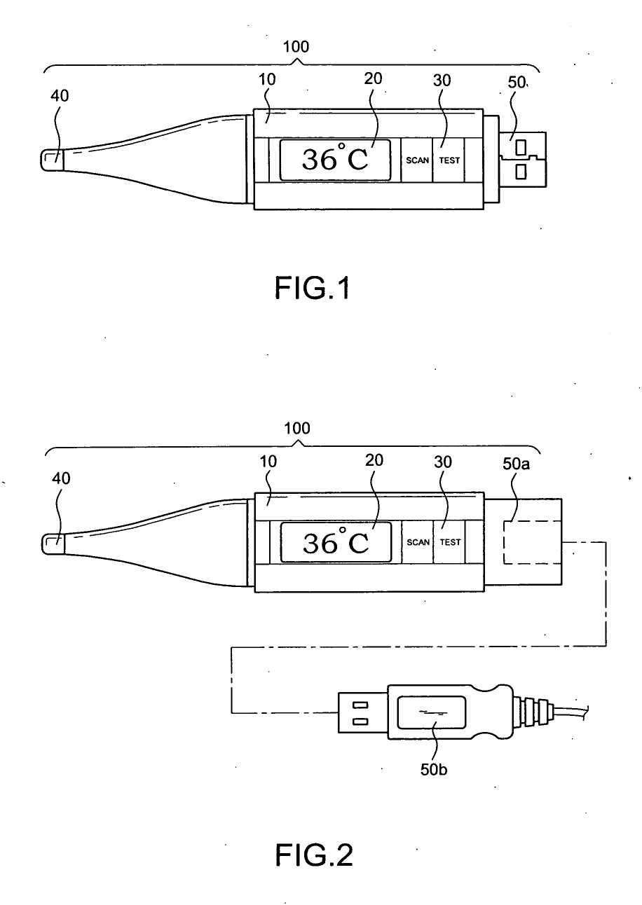

Referring to FIG. 1, a clinical thermometer 100 in accordance with a first preferred embodiment of the present invention comprises:

a casing 10, being substantially in a rectangular shape for facilitating users to hold the thermometer 100 and having a containing space (not shown in the figure) for installing an LCD device 20 and a press key unit 30;

a body temperature detecting element 40, installed at a small end of the casing 10 for detecting a body temperature; and

a USB interface 50, installed at another side of the casing 10 and corresponding to the body temperature detecting element 40, and the USB interface 50 of this preferred embodiment is in the form of a male connector protruded from a side of the casing.

Referring to FIG. 2 for the second preferred embodiment of the present invention, the elements of this embodiment use the same numeric symbols as those illustrated in the first preferred embodiment, and their difference resides on that the USB interface 50a is in the form of a female connector concavely disposed at a side of the casing 10. When the clinical thermometer 100 of this embodiment is connected to a personal computer, a USB cable 50b is used. Regardless of the first or second preferred embodiment, both embodiments are provided for facilitating a quick connection to the personal computer.

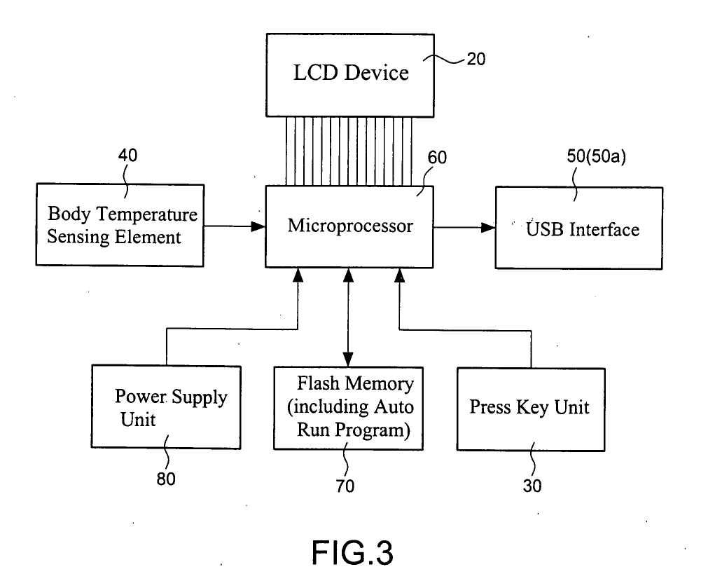

Referring to FIG. 3 for the circuit block diagram of the present invention, the invention comprises an LCD device 20 disposed at a surface of a casing 10, a press key unit 30 and a body temperature detecting element 40, and further comprises:

a microprocessor 60, for receiving a signal transmitted from the body temperature detecting element 40, computing the body temperature, and displaying the numeric data (such as 36° C.) of the body temperature by the LCD device 20;

a flash memory 70, installed in the casing 10 and coupled to the microprocessor 60 for storing the data, and having a partitioned memory block with at least one area for storing an Auto Run program; and

a power supply unit 80, installed in the casing 10 and electrically connected to the microprocessor 60 for supplying electric power required by each of the foregoing components, and having one or two batteries.

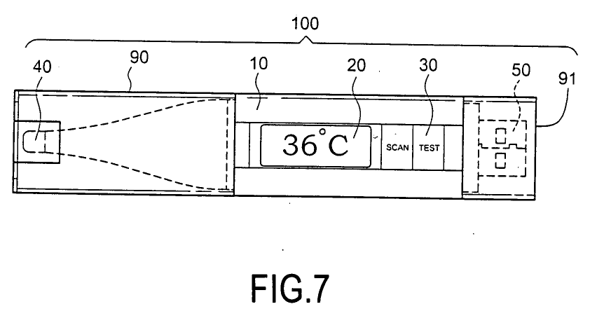

Referring to FIG. 7, the casing 10 of the invention includes a first protective cover 90 disposed on a side of the casing 10 with the body temperature detecting element 40 and a second protective cover 91 disposed on another side of the casing 10 without the body temperature detecting element to prevent the casing 10 from being damaged.

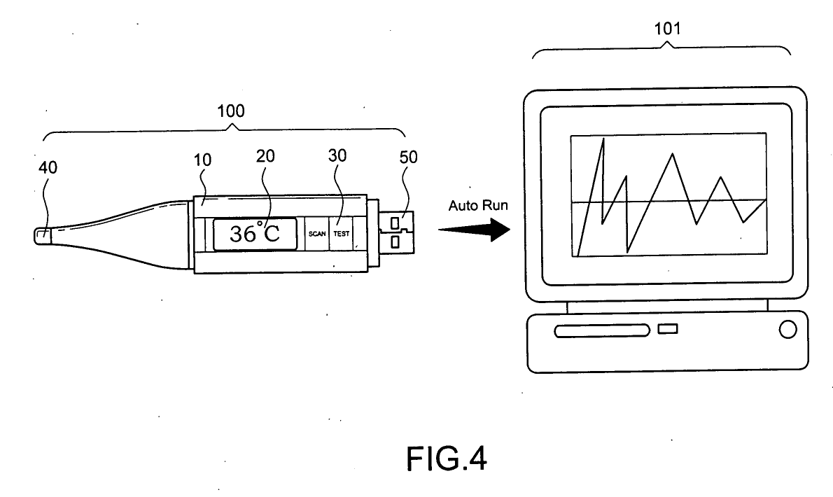

The foregoing USB interface 50, 50a disposed at a side of the casing 10 is electrically connected to the microprocessor 60, such that when the USB interface 50 as shown in FIG. 4 is connected to a personal computer 101, the Auto Run program pre-stored in the flash memory 70 automatically sends the body temperature data accumulated by a user in a predetermined period such as a week, a month or a year to the personal computer 101 and adopts the original software, hardware and operating system of the personal computer 101 for computing the data. If the clinical thermometer 100 of the invention is connected to the personal computer 101, the data can be sent automatically and the application is very convenient. Therefore, users can obtain the information about their body temperature change and use a graphical analysis to predict the best day of getting pregnant or the safe period for contraception.

In summation of the technical characteristics described above, the present invention has the following advantages:

1. The clinical thermometer 100 of the present invention comes with automatic recording and storing functions, so that users just need to measure their body temperature at a fixed time each day without the need of recording the data by pen and paper, and thus its application is very convenient.

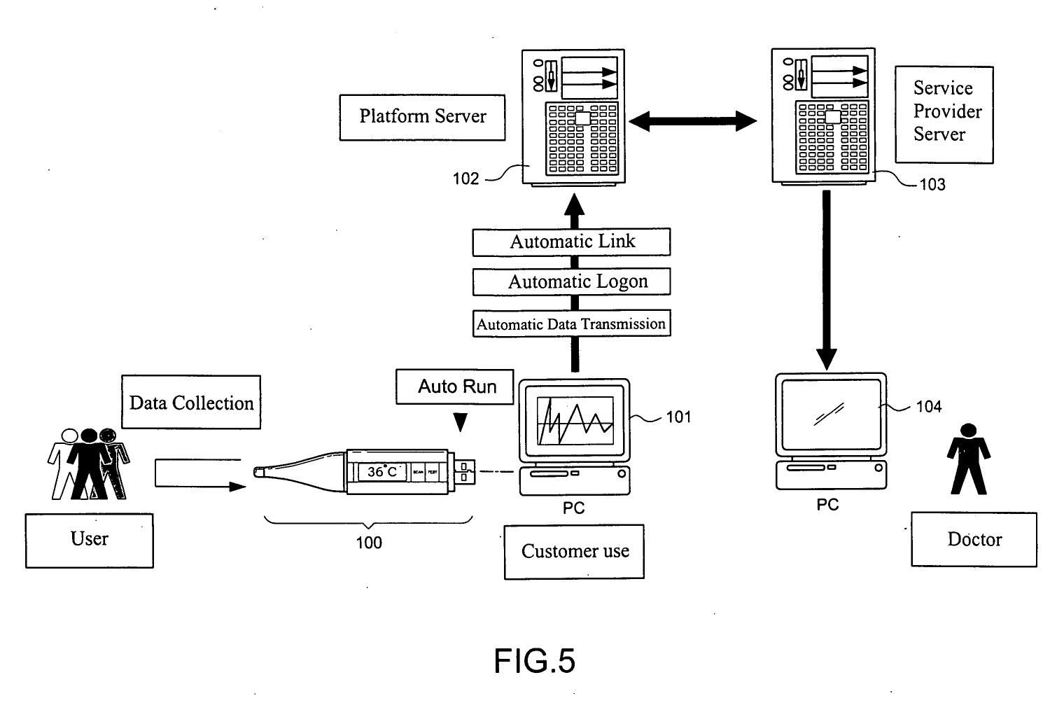

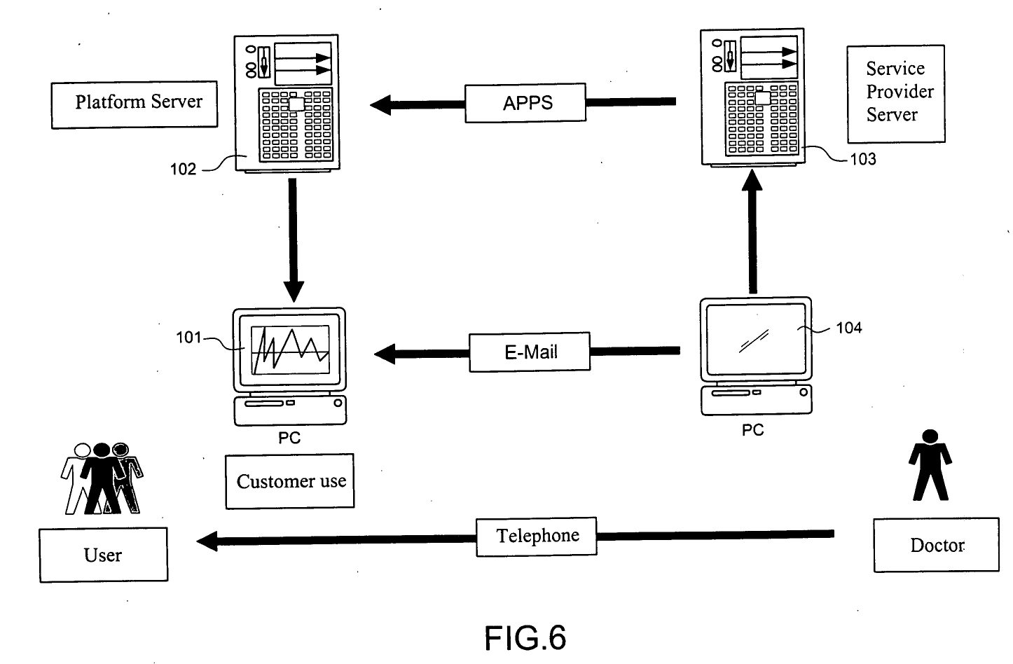

2. The present invention uses the Auto Run program stored in the flash memory 70, such that when the clinical thermometer 100 is connected to the personal computer 101, the body temperature data measured daily in a certain period of time will be displayed on the personal computer 101 to facilitate users to understand their physiological conditions. Further, the user data can be obtained as shown in FIG. 5, and the personal computer 101 automatically sends data, logs on, and links to a platform server 102 and connected to a service provider server 103, such that a doctor at a remote end can obtain the user's physiological information through the personal computer 101, so as to perform a periodical tracking and give medical advices. In FIG. 7, the doctor can analyze and evaluate the conditions and inform the users through a telephone or an E-mail or the data can be uploaded to the service provider server 103, so that the users can obtain the doctor's analysis reports and medical advices from their personal computer 101 through the platform server 102, and its application is very convenient and practical.

Many changes and modifications in the above-described embodiments of the invention can, of course, be carried out without departing from the scope thereof. Accordingly, to promote the progress in science and the useful arts, the invention is disclosed and is intended to be limited only by the scope of the appended claims.

What is claimed is:

1. A clinical thermometer capable of recording and analyzing the day of getting pregnant, comprising:
a) a casing being substantially in a rectangular shape and having a containing space for installing an LCD device and a press key unit at the front side of the casing;
b) a body temperature detecting element installed at a small end of the casing for detecting a body temperature;
c) a microprocessor for receiving a signal transmitted from the body temperature detecting element and computing the body temperature and displaying a numeric data of the body temperature by the LCD device;
d) a flash memory coupled to the microprocessor for storing the data and having a partitioned memory block with at least one area for storing an Auto Run program;
e) a power supply unit electrically coupled to the microprocessor for supplying electric power required by each of the foregoing elements; and
f) a USB interface installed at a side of the casing and electrically coupled to the microprocessor,
whereby, when the USB interface is coupled to a personal computer, the Auto Run program pre-stored in the flash memory can automatically send the body temperature data accumulated by a user for a predetermined time to an operating system of the personal computer, so as to facilitate recording and analyzing a better day of getting pregnant.

2. The clinical thermometer capable of recording and analyzing the day of getting pregnant as recited in claim 1, wherein the USB interface is in the form of a male connector protruded from a side of the casing.

3. The clinical thermometer capable of recording and analyzing the day of getting pregnant as recited in claim 1, wherein the USB interface is in the form of a female connector concavely disposed at a side of the casing.

4. The clinical thermometer capable of recording and analyzing the day of getting pregnant as recited in claim 1, wherein the power supply unit includes a battery.

5. The clinical thermometer capable of recording and analyzing the day of getting pregnant as recited in claim 1, wherein the casing includes a first protective cover and a second protective cover disposed respectively on both sides of the casing.
